(19) United States
(12) Patent Application Publication
(10) Pub. No.: US 2022/0015934 A1
(43) Pub. Date: Jan. 20, 2022

(54) Apparatus for stabilizing body joints and/or supporting items of sports equipment

(71) Applicant: BETTERGUARDS TECHNOLOGY GmbH, BERLIN (DE)

(72) Inventors: Vinzenz BICHLER, Berlin (DE); Timo STUMPER, Berlin (DE); Oscar BUSCHINGER, Berlin (DE)

(73) Assignee: BETTERGUARDS TECHNOLOGY GmbH, BERLIN (DE)

(21) Appl. No.: 17/311,597

(22) PCT Filed: Dec. 5, 2019
(86) PCT No.: PCT/EP2019/083856

§ 371 Date: Jun. 7, 2021

(30) Foreign Application Priority Data
Dec. 7, 2018 (DE) 10 2018 131 457.4

(51) Int. Cl. A61F 5/01; F16F 9/19; F16F 9/32; F16F 9/512; F16F 9/516

(52) U.S. Cl. CPC A61F 5/0104; F16F 9/19; A61F 2005/0197; F16F 9/5126; F16F 9/516; F16F 9/3214

(57) ABSTRACT

Disclosed is an apparatus for stabilizing body joints and/or for supporting items of sports equipment, comprising: a receptacle, wherein the receptacle is filled with a filling medium, a first body for interacting with the filling medium, wherein the first body is displaceably arranged in the receptacle, a force transmission body for transmitting an external force to the first body, a second body for interacting with the filling medium, which is displaceably arranged in the receptacle, wherein the second body is elastically coupled to the first body via a coupling element, wherein the second body and/or the first body has at least one passage opening, through which the filling medium can flow, and wherein the first body forms a valve body and the second body forms a valve seat, such that a flow of the filling medium through the passage opening can be permitted or inhibited as a function of the valve position, wherein, in a displacement direction relative to the first body, the second body overlaps therewith.

### CROSS REFERENCE TO RELATED APPLICATIONS

This application is a national stage U.S. patent application of International Application No. PCT/EP2019/083856, filed on Dec. 5, 2019, and claims foreign priority to German Patent Application No. DE 10 2018 131 457, filed on Dec. 7, 2018 the entirety of each of which is incorporated herein by reference.

### TECHNICAL FIELD

The present disclosure relates to a device for stabilizing body joints or for supporting items of sports equipment, which device comprises a receptacle which is filled with a filling medium, a first body for interacting with the filling medium, the first body being arranged displaceably in the receptacle, and a means for transmitting an external force to the first body.

### DESCRIPTION OF THE RELATED ART

The practice of stabilizing body joints, muscles and tendons by means of devices which allow an adaptive movement restriction is known. The provision of items of sports equipment which may be exposed to backward movements with adaptive movement restricting devices is also known.

The adaptive behavior of such devices is achieved, inter alia, by virtue of two bodies moving relative to one another, with a filling medium being located between the bodies. In this respect, a body of the device can form a receptacle which is filled with the filling medium. The other body may form a pull-out body, which is arranged movably in the receptacle. The filling medium can flow in the region between the receptacle and the pull-out body when the two bodies move relative to one another. The flow velocity of the filling medium significantly depends on the cross-sectional area perpendicular to a relative displacement direction of the receptacle and of the pull-out body. This cross-sectional area, which is available for the flow of the filling medium, is also referred to as the hydraulic diameter and is ultimately crucial for the reactive behavior of the device in the event of an external action of force. In this way, the selection of the hydraulic diameter makes it possible to establish the resistance with which the device counteracts external forces. The devices can be fixed between two locations on the body of a user or two elements of an item of sports equipment that can be moved relative to one another.

If physiological forces, that is to say forces which are not critical for the body part or component correspondingly to be stabilized, are introduced into the device via the two locations on the body of the user, a corresponding relative movement of the receptacle and of the pull-out body and thus a movement of the body part to be stabilized is permitted in accordance with the hydraulic diameter in the device.

If, by contrast, unphysiological forces, that is to say forces which are critical for the body part or component correspondingly to be stabilized, are introduced into the device, a relative movement between the pull-out body and the receptacle is only possible with a very high expenditure of force on account of the hydraulic diameter.

EP 3 238 670 A1 discloses a device for stabilizing body joints that allows an adaptive behavior depending on the intensity of an acting force.

The device comprises a receptacle which is filled with a filling medium, a first body for interacting with the filling medium, the first body being arranged displaceably in the receptacle, and a force transferring means for transferring an external force to the first body. A second body for interacting with the filling medium is arranged displaceably in the receptacle, the second body being elastically coupled to the first body by way of a coupling element. The second body also has a passage opening, through which the filling medium can flow. The passage opening in the second body provides a hydraulic diameter for the filling medium, through which the filling medium can flow provided that the first body and the second body are spaced apart from one another.

In this case, the first body forms a valve body and the second body forms a valve seat, with the result that a flow of the medium through the passage opening can be permitted or prevented depending on the valve position.

External forces which act on the first body can be transferred to the second body by way of the coupling element. Correspondingly, the first body is able to push and/or pull the second body through the filling medium by means of the coupling element for this purpose.

In this respect, the coupling element is configured in such a way that, when an external force acts on the first body, in the region of a physiological velocity, it transfers a force to the second body, with the result that said second body can be moved through the filling medium together with the first body.

If the force acting on the second body by way of the first body and the coupling element leads to critical relative displacement velocities in the device, that is to say to unphysiological velocities, the coupling element yields, as a result of which the first body moves toward the second body. As a result, the hydraulic diameter is reduced until the valve formed by the two bodies is closed.

If a hydraulic diameter through which the filling medium can flow is no longer available, the first body and the second body cannot be moved further through the filling medium. The device becomes blocked.

It has been found that there is a need for comparatively still shorter reaction times until the complete blockage of a movement, in the case of which blockage the device described above reaches its limits.

It has also been found that the device described above cannot always guarantee that the passage opening is closed in a satisfactory manner. This can lead to irregularities in the behavior of the device.

### SUMMARY OF THE DISCLOSURE

The present disclosure provides an device for stabilizing body joints or for supporting items of sports equipment.

Described is a device for stabilizing body joints or for supporting items of sports equipment, said device comprising a receptacle, the receptacle being filled with a filling medium, a first body for interacting with the filling medium, the first body being arranged displaceably in the receptacle, a force transferring body for transferring an external force to the first body, a second body for interacting with the filling medium that is arranged displaceably in the receptacle, the second body being elastically coupled to the first body by way of a coupling element, the second body and/or the first body having at least one passage opening, through which the filling medium can flow, and the first body forming a valve body and the second body forming a valve seat, with the result that a flow of the filling medium through the passage opening can be permitted or prevented depending on the valve position. According to the disclosure, the second body overlaps the first body in a relative displacement direction with respect thereto.

An overlap of the first body and the second body in a relative displacement direction makes it possible to guide the two bodies when they are moved relative to one another on account of an external action of force. As a result, the likelihood may be increased that a contacting of the two bodies, which results in the passage opening becoming blocked, the passage opening is completely closed. This contributes to a uniform behavior of the device.

The relative displacement direction denotes the direction in which the first body and the second body can be moved relative to one another owing to an external action of force and/or the coupling element.

In an implementation, the second body comprises at least one projection in the relative displacement direction, the projection overlapping the first body with respect to the relative displacement direction independently of a relative displacement of the first body and of the second body with respect to one another.

As a result, it is possible to always provide a contact, i.e. a guide of the first body on the second body. Consequently, there is an overlap of the projection of the second body with the first body both when the device is located in a starting position, in which no external force acts on the device and in particular on the first and the second body, and when the device is located in a blocking position, in which the first body rests on the second body and in the process closes the passage opening.

In another embodiment, the second body has at least one surface extending in the relative displacement direction, in order to guide the first body in the relative displacement direction. The guide surface may butt against the first body and make contact therewith along a complete displacement travel, i.e. between the starting position and the blocked position of the device.

In one refinement, the first body and/or the second body have/has at least one undercut with respect to the respective other body, in order to delimit a relative displacement distance between the two bodies. The relative displacement distance between the first body and the second body directly influences the reaction time of the device. The selection of a suitable relative displacement distance provides a further possible setting of the reaction time of the device in addition to the coupling element.

An undercut contributes to the fact that the first body and the second body not only overlap, but at least partially back-engage. The undercut of the two bodies may be provided in a different form. An undercut may be arranged at a point of an overlapping portion of the second body that is outermost with respect to the relative displacement direction, for example. As an alternative, both the first body and the second body may each have an undercut, the undercuts defining a starting position of the first body and of the second body in the starting position of the device.

In an implementation, the projection of the second body defines a receiving space, in which the first body is at least partially received, the receiving space delimiting a relative displacement distance of the first body and of the second body in relation to one another in the relative displacement direction. The receiving space makes it possible to provide both a guided relative movement between the first body and the second body and a delimitation of the maximum relative displacement distance between the first body and the second body.

The receiving space may have one or more openings. The first body may thus protrude in part from the receiving space, for example. In the latter case, only part of the first body is permanently received in the receiving space, while another part of the first body protrudes from the receiving space depending on a relative displacement position of the two bodies in relation to one another. As a result, it is possible to provide a guidance of the first body and of the second body in relation to one another.

In another embodiment, the projection comprises one or more recesses, which are fluidically connected to the passage opening, in order to allow a flow of the filling medium relative to the second body, preferably through the second body, in an open valve position. As a result, the possibility that the filling medium can flow inside the receptacle relative to the first body and in particular the second body can be maintained until the first body blocks the passage opening in the second body.

The at least one recess may also perform a mounting function. For this, the recess may be configured in such a way that the first body can be inserted through the recess into the receiving space of the second body.

According to an implementation, the second body and/or the first body subdivide(s) a cavity of the receptacle into a first chamber and a second chamber. If the first body and the second body move through the receptacle on account of the action of an external force when the valve position is open, the filling medium may flow through the passage opening in the second body from the first chamber into the second chamber.

In a configuration, the coupling element is preloaded between the first body and the second body in a starting position of the device, the first body being in contact with the second body.

In the devices known according to the prior art, which are designed without preloading of the coupling element, for example of a spring, coupling elements with comparatively high resistance properties, such as a high spring strength, lead to comparatively long closing distances. The latter can adversely affect the reproducibility of the behavior of the device.

The lack of preloading of the coupling element also leads to the first body being moved toward the second body already when comparatively low forces are acting, as a result of which the potential flow path of the operative medium toward the passage opening is also already reduced. This can lead to an imprecise, non-reproducible behavior of the device when comparatively low external forces are acting on the first and/or the second body.

The preloading of the coupling element makes it possible to use a coupling element with low forces of resistance. In that case, the preloading of the coupling element may be selected such that the coupling element does not yet react when comparatively low external forces are acting and there is no relative movement between the first and the second body. Overall, the preloading of the coupling element makes it possible to increase a reaction threshold with respect to the external forces acting on the device.

In addition, the preloading of the coupling element makes it possible to realize shorter relative displacement distances between the first body and the second body. In other words, the maximum travel between the first body and the second body can be reduced by preloading of the coupling element. A shorter maximum relative displacement distance between the first body and the second body makes it possible to significantly reduce the reaction distance and thus the reaction time until a sufficiently high force of resistance of the device has built up. Finally, the preloading of the coupling element also influences the component dimension of the device, and in this way the overall length of the device can be reduced by the shortening of the relative displacement distance.

### BRIEF DESCRIPTION OF THE FIGURES

Further embodiments of the disclosure will be explained in more detail by the following description of the figures, in which:

FIG. 1A schematically shows a perspective view of a device for stabilizing body joints and/or items of sports equipment,

FIG. 1B schematically shows a sectional view from the side of the device from FIG. 1A in a starting state,

FIG. 2A schematically shows a view of a detail of the device according to FIG. 1B in a starting position,

FIG. 2B schematically shows a view of a detail of the device according to FIG. 1B in a blocking position,

FIG. 3 schematically shows a perspective view of a detail of a second body,

FIG. 4A schematically shows a view from the side of a detail of the first and second bodies,

FIG. 4B schematically shows a rear view of the first and second bodies as per FIG. 4A,

FIG. 5A schematically shows a view of a detail of an alternative embodiment of the first and second bodies,

FIG. 5B schematically shows a view of a detail of an alternative embodiment of the first and second bodies,

FIG. 6 schematically shows a view of a detail of an alternative embodiment of the first and second bodies,

FIG. 7A schematically shows a view of a detail of an alternative embodiment of the first and second bodies, and

FIG. 7B schematically shows a view from the side of a detail of the embodiment as per FIG. 7A.

### DETAILED DESCRIPTION

In the following text, exemplary embodiments are described with reference to the figures. In the figures, elements which are the same, similar or have the same effect are provided with identical reference signs in the different figures, and a repeated description of these elements is in part omitted to avoid redundancies.

Various embodiments now will be described more fully hereinafter with reference to the accompanying drawings, which form a part hereof, and which show, by way of illustration, specific embodiments by which the innovations described herein can be practiced. The embodiments can, however, be embodied in many different forms and should not be construed as limited to the embodiments set forth herein; rather, these embodiments are provided so that this disclosure will be thorough and complete, and will fully convey the scope of the embodiments to those skilled in the art. The following detailed description is, therefore, not to be taken in a limiting sense.

Throughout the specification and claims, the following terms take the meanings explicitly associated herein, unless the context clearly dictates otherwise. The term “herein” refers to the specification, claims, and drawings associated with the current application. The phrase “in an embodiment” as used herein does not necessarily refer to the same embodiment, though it can. Furthermore, the phrase “in another embodiment” as used herein does not necessarily refer to a different embodiment, although it can. Thus, as described below, various embodiments can be readily combined, without departing from the scope or spirit of the invention.

In addition, as used herein, the term “or” is an inclusive “or” operator and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The function of the present device is described below with reference to use in the sports sector. In this respect, the device for damping the movement of two points on a body that can move relative to one another is used. Such a body may be a sports shoe, for example, which coupled with the present device can counteract a bending-over movement. The present device is not limited to the areas of use described in this document, however. Thus, it can also be arranged between two body parts of a living being, in order to damp a corresponding body movement. As an alternative, the device can also be used in other everyday articles, in which the intention is to damp abruptly increasing forces between a body or an article.

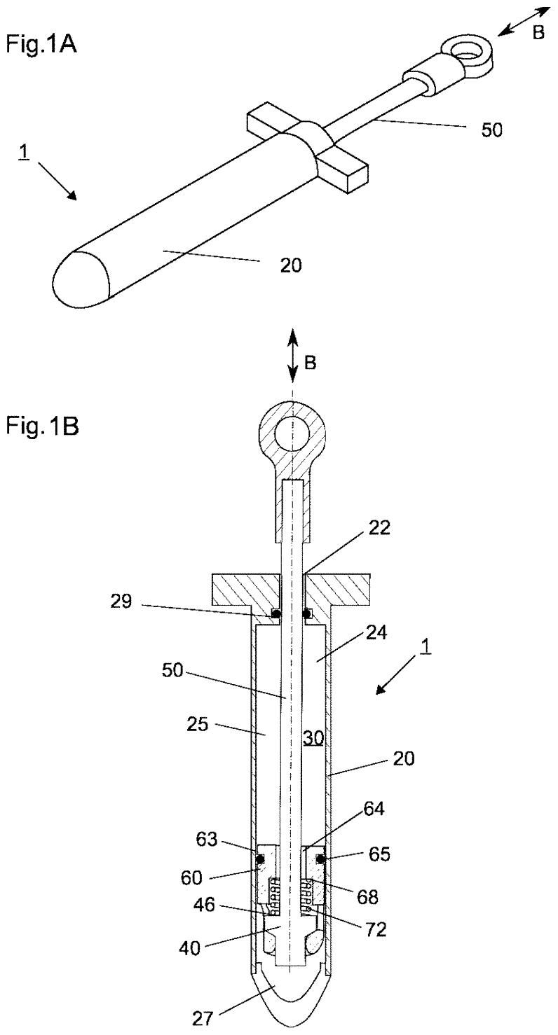

FIG. 1A shows a perspective view of a device 1 for stabilizing body joints and/or items of sports equipment. A force transferring means 50 projects from a cylindrical receptacle 20. In this respect, the receptacle 20 may be fastened to a point on the body of a user or an item of sports equipment, such as a sports shoe. That end of the force transferring means 50 which lies outside the device 20 may be fastened to a second point on the body. The first point on the body and the second point on the body are distinguished in that they are exposed to abrupt relative movements with respect to one another. The direction B represents the movement direction of the device. As an alternative, the receptacle may also have a cuboidal form.

FIG. 1B shows a sectional view of the device 1, which is in a starting state. The receptacle 20 has an opening 22, through which a force transferring means 50 protrudes into the interior space of the device 20. If the point on the body to which the receptacle 20 is fastened moves relative to the point on the body on which the force transferring means 50 is arranged, the force transferring means 50 moves relative to the receptacle 20. In particular, the force transferring means 50 may move in a movement direction B further into the receptacle 20 or further out of the receptacle 20.

The receptacle 20 of the device 1 is manufactured from stainless steel. As an alternative, the receptacle may also be manufactured from plastic. Fiber-reinforced plastics, inter alia, may also be used. As an alternative, the receptacle may also be manufactured from other metals, such as aluminum or magnesium. In addition, the receptacle may also be manufactured from ceramic. The force transferring means 50 is formed by a cable made of wire and extends from a first body 40 through a passage opening 64 in a second body 60 and finally through the opening 22 in the receptacle 20. As an alternative, the force transferring means 50 may be designed in the form of a rod element made of plastic. Further, the force transferring means may also be designed in the form of a fiber. Furthermore, the force transferring means may also be manufactured from metal, such as aluminum, magnesium or stainless steel.

An interior space 24 of the device 20 is filled with a filling medium 30. The filling medium 30 is a Newtonian fluid, such as silicone oil. As an alternative, dilatant fluids may also be used as filling medium. In addition, a shear-thickening plastic may also be used. In this case, the plastic is present in powder form. Furthermore, sand may also be used as filling medium.

In addition, arranged in the interior space 24 of the device 20 is a first body 40, which can be moved through the filling medium 30 in the movement direction B relative to the receptacle 20. In the present embodiment, the first body 40 is connected to the force transferring means 50 in one piece. As an alternative, the first body may be coupled to the force transferring means in a force transferring region, with the result that a force starting from the force transferring means can be transferred to the first body.

The cross section of the first body 40 is smaller than the cross section of the receptacle 20, with the result that the filling medium 30 can flow in the movement direction B relative to the first body 40. The distance between the first body and the receptacle 20 forms a hydraulic diameter.

The first body 40 is manufactured from plastic. As an alternative, the first body may also be manufactured from a metal such as aluminum, magnesium or steel.

In addition, arranged in the interior space 24 of the receptacle 20 is a second body 60, which can be moved relative to the receptacle 20 in the movement direction B.

The second body 60 is manufactured from plastic. As an alternative, the second body may also be manufactured from a metal, such as aluminum. The second body has a peripheral groove 63 for receiving an O-ring 65. The second body 60 subdivides the interior space 24 of the receptacle 20 into a first chamber 25 and a second chamber 27. The O-ring 65 prevents the filling medium 30 from being able to flow along an external peripheral surface of the second body 60 between the first chamber 25 and the second chamber 27.

The first body 40 is coupled to the second body 60 by way of an elastic coupling element 70. The elastic coupling element shown in FIG. 1B is formed by a spring 72, which is mounted at one end in a spring seat 46 of the first body 40 and at the other end in a spring seat 68 of the second body 60. The second body 60 may be pushed over the spring 72 by means of the first body 40. In this respect, the spring 72 may be manufactured from plastic or from metal. As an alternative, the elastic coupling element may also be designed in the form of an elastic polymer or rubber.

In a further alternative, the first body, the second body and the elastic coupling element are injection molded in one piece.

The second body 60 also comprises a passage opening 64, through which the filling medium 30 can flow. If the second body 60 moves relative to the receptacle 20 in accordance with a force starting from the coupling element, the filling medium 30 can flow through the passage opening 64 in the second body 60 between the first chamber 25 and the second chamber 27. The passage opening 64 correspondingly defines a hydraulic diameter for the filling medium 30 in the region of the second body 60.

The device 1 shown in FIGS. 1A and 1B is subjected to tensile loading, i.e. to loading which results from the point on the body to which the receptacle 20 is fastened and the point on the body to which the force transferring means 50 is fastened being moved away from each other. If the force transferring means 50 is pulled out of the device 20, it pulls the first body 40 with it, as a result of which it presses onto the second body 60 by means of the coupling element 70. The force transferring means 50 extends from the first body 40 through the passage opening 64 in the second body 60 and finally through the opening 22 in the receptacle 20. Arranged in the region of the opening 22 are sealing inserts 29 which seal off the interior space 24 of the receptacle 20 with respect to the surroundings, and therefore the filling medium 30 can be kept in the interior space 24 of the receptacle 20.

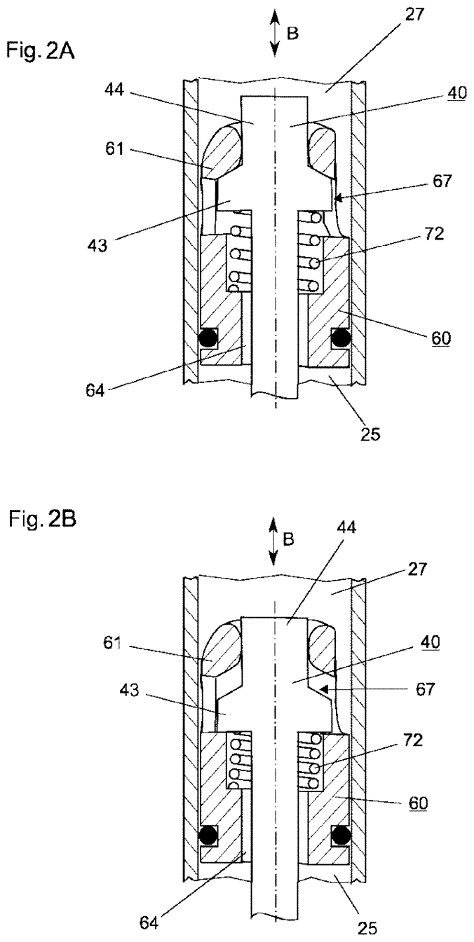

FIGS. 2A and 2B show views of a detail of the device, with reference to which the interaction of the first body 40 with the second body 60 is described in more detail. The second body 60 has a projection 61, which extends toward and overlaps the first body 40. The projection 61 is configured to engage behind the first body 40. In this respect, the projection 61 defines a receiving space 67, in which the first body 40 is partially received.

According to the present embodiment, the first body 40 has a stepped construction. Thus, the first body 40 has a first portion 43, which is received displaceably in the receiving space 67 of the second body 60, and a second portion 44, the diameter of which tapers with respect to the first portion 43 and which extends out of the receiving space in the direction of the movement direction B. The projection 61 is configured in such a way that it engages behind the first portion 43 of the first body 40 and forms a guide in the movement direction B for the second portion 44. In this way, the first body can be centered with respect to the second body, with the result that a closing of the passage opening 64 can be ensured when the first body 40 and the second body 60 assume a closed valve position.

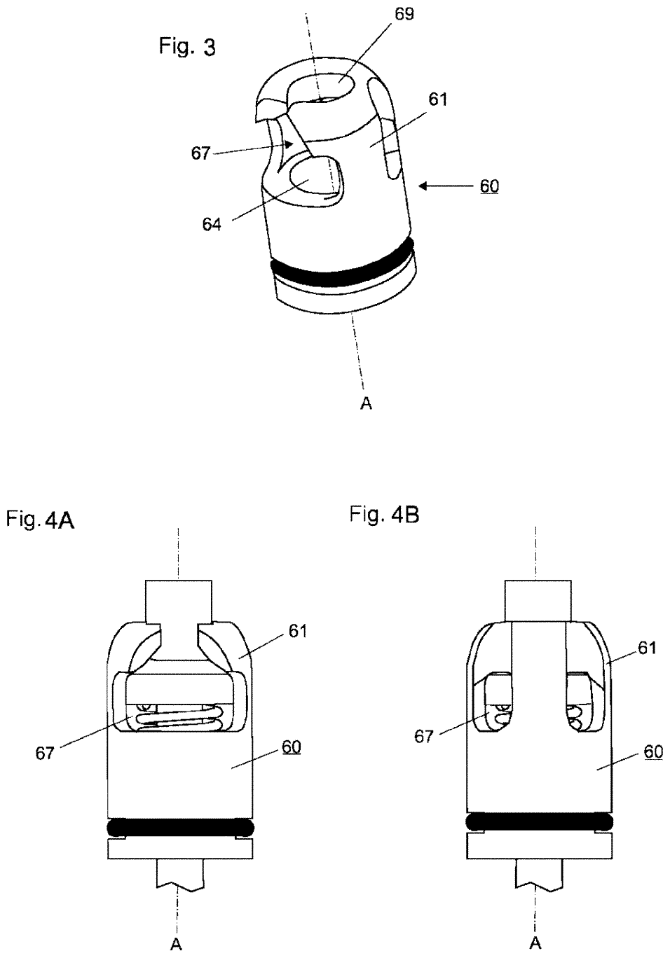

FIG. 3 shows a perspective view of the second body 60. The projection 61 continues in an axial direction A of the second body 60. The projection 61 is curved inwardly toward the axis at an end region thereof, as a result of which an undercut for the first portion of the first body to engage behind is formed. The projection 61 defines a receiving space 67 for receiving the first portion of the first body. The receiving space 67 adjoins the passage opening 64. At that end of the receiving space 67 which is situated opposite the passage opening 64, the undercut of the projection 61 defines an opening, which serves as a guide 69 for the second portion of the first body. The second portion of the first body may partially leave/enter the receiving space 67 in the relative displacement direction of the first body and of the second body relative to one another.

With respect to the longitudinal axis A of the second body 60, the projection 61 has a plurality of lateral interruptions, as can also be seen in FIGS. 4A and 4B. These fulfil 2 tasks. Firstly, a lateral interruption allows the first body to be inserted into the receiving space 67 of the second body 60. The projection 61 has a correspondingly flexible configuration, such that the first body can be clipped into the receiving space 67. Secondly, as can be seen in FIGS. 2A and 2B, the interruptions allow flow paths for the filling medium 30 between the second chamber 27 and the passage opening 64.

FIG. 2A shows the starting position of the device for damping a movement. The first portion 43 of the first body 40 is in contact with the undercut of the projection 61 of the second body 60. This starting position may be referred to as the open valve position of the device.

In the starting position of the device 1 shown in FIG. 2A, the spring 72 is subject to preloading. The preloading of the spring 72 is selected such that, when comparatively low external forces are acting, the spring 72 does not yet react and there is no relative movement between the first body 40 and the second body 60. Overall, the preloading of the spring 72 allows a higher reaction threshold with respect to the external forces acting on the device 1.

In addition, the preloading of the spring makes it possible to realize shorter relative displacement distances between the first body 40 and the second body 60. In other words, the maximum travel between the first body 40 and the second body 60 can be reduced by preloading of the spring 72. A shorter maximum relative displacement distance between the first body 40 and the second body 60 makes it possible to significantly reduce the reaction distance and thus the reaction time until a sufficiently high force of resistance of the device 1 has built up.

In the following text, the function of the device is described with reference to FIGS. 2A and 2B. If a force acts in the region of a physiological velocity on the force transferring means 50 such that the first body 40 is pulled in the direction of the second body 60, the second body 60 is also conjointly moved by means of the spring 72. The interaction between the second body 60 and the filling medium 30 leads to a force of resistance that acts on the second body 60. The force of resistance is transferred to the spring 72 by the second pull-out body 60. If the spring 72 were not subject to preloading, it would begin immediately with a compression movement. In the present case, however, the spring 72 is subject to preloading, which can be attributed to the arrangement of the first body 40 in the receiving space 67 of the second body 60. In other words, the projection 61 of the second body 60 holds the first body 40 in a preloaded starting position with respect to the second body 60.

The strength of the spring 72 is selected in such a way that the preloaded spring 72 is compressed when a threshold value, of the force of resistance acting on the second body 60, has been reached. At a force of resistance below the threshold value, there is no relative movement between the first body and the second body. At a force of resistance above the threshold value, the spring begins to compress, as a result of which the first body 40 moves toward the second body until the passage opening 64 is completely closed, as shown in FIG. 2B. In this state, the hydraulic diameter of the device is zero. Consequently, there is no longer any hydraulic diameter, through which the filling medium 30 can flow between the first chamber 25 and the second chamber 27.

The selection of comparatively low spring strengths makes it possible, in combination with the preloading of the spring, to realize a comparatively quick reaction time of the device, i.e. a quick closing of the passage opening 64. A flow of the filling medium from the first chamber into the second chamber is no longer possible, and therefore the two pull-out bodies 40, 60 can no longer be moved relative to the receptacle 20. In this state, the device 1 permits no further movement between two points on the body to be supported/damped.

For example, the threshold value may be 4.5 N and the system may immediately completely close when a force of resistance jumps from 4.5 N to 5 N.

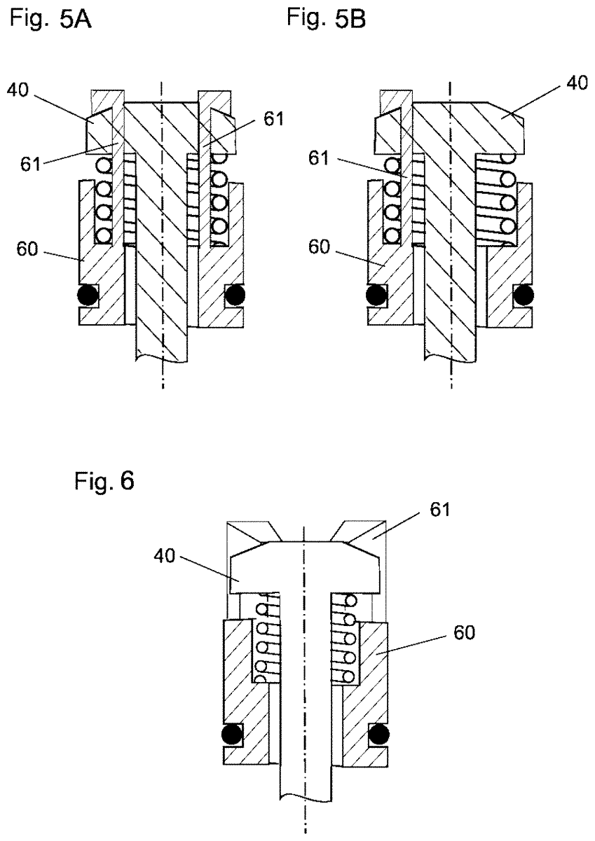

FIGS. 5A to 7B show alternative embodiments of the first and second bodies. FIG. 5A shows a second body 60 with projections 61, which lead through the first body 40. Arranged at the end of the projections 61 are undercuts, for example in the form of flanges, which allow a back-engagement of the first body 40.

FIG. 5B differs from FIG. 5A in that the second body 60 has only one projection 61, which is guided through the first body 40 to engage behind it.

FIG. 6 shows a second body 60 with hook-shaped projections 61, which are configured to engage behind the first body 40. The projections 61 bypass the first body 40 at the side in this case.

FIGS. 7A and 7B show a cylindrical second body 60, in which a cylindrical first body 40 may at least partially be received. The second body 60 has recesses which form a guide 69, in order to guide projections of the first body 40 therein. FIGS. 7A and 7B show a starting situation of the device, in which the lateral projections of the first body 40 butt against a first end of the recesses. FIG. 7A shows that the first body 40, as described above, is kept in the starting position by means of a spring 72. As shown in FIG. 7B, the cylindrical second body 60 has lateral recesses 62, which provide a hydraulic diameter, through which the filling medium can flow past the first body 40 and toward the passage opening 64 in the second body 60.

The functional principle of the embodiments according to FIGS. 5A to 7B is the same as the functional principle according to FIGS. 2A and 2B described above.

If applicable, all individual features illustrated in the exemplary embodiments may be combined with one another and/or interchanged with one another without departing from the scope of the disclosure.

### LIST OF REFERENCE SIGNS

1 Device
20 Receptacle
22 Opening
24 Interior space
25 First chamber
26 Interior surface
27 Second chamber
28 Step
29 Sealing insert
30 Filling medium
40 First body
43 First portion
44 Second portion
46 Spring seat
50 Force transferring means
60 Second body
61 Projection
62 Recess
63 Groove
64 Passage opening
65 O-ring
66 Guide projection
67 Receiving space
68 Spring seat
69 Guide
70 Coupling element
72 Spring
A Axial direction
B Movement direction

1. A device for stabilizing body joints and/or for supporting items of sports equipment, said device comprising:
a receptacle, wherein the receptacle is filled with a filling medium,
a first body for interacting with the filling medium, wherein the first body is arranged displaceably in the receptacle,
a force transferring body for transferring an external force to the first body,
a second body for interacting with the filling medium (30), which is arranged displaceably in the receptacle,
wherein the second body is elastically coupled to the first body by way of a coupling element,
wherein the second body and/or the first body have at least one passage opening (64), through which the filling medium can flow, and
wherein the first body forms a valve body and the second body forms a valve seat, with the result that a flow of the filling medium through the passage opening can be permitted or prevented depending on the valve position,
wherein the second body overlaps with the first body in a relative displacement direction to the first body.

2. The device as claimed in claim 1, wherein the second body comprises at least one projection in the relative displacement direction, wherein the projection overlaps the first body with respect to the relative displacement direction independently of a relative displacement of the first body and of the second body with respect to one another.

3. The device as claimed in claim 1, wherein the first body and/or the second body has at least one undercut with respect to the respective other body that is configured to delimit the relative displacement distance between the two bodies.

4. The device as claimed in claim 1, wherein the second body has at least one guide surface extending in the relative displacement direction that is configured to guide the first body in the relative displacement direction.

5. The device as claimed in claim 1, wherein the projection of the first body defines a receiving space in which the first body is at least partially received, and the receiving space delimits a relative displacement distance of the first body and the second body in relation to one another in the relative displacement direction.

6. The device as claimed in claim 1, wherein the projection comprises one or more interruptions that are fluidically connected to the passage opening and configured to allow a flow of the filling medium relative to the second body.

7. The device as claimed in claim 1, wherein the second body and/or the first body subdivide a cavity of the receptacle into a first chamber and a second chamber.

8. The device as claimed in claim 7, wherein the passage opening can provide a flow of the fluid medium between the first chamber and the second chamber in an open valve position.

9. The device as claimed in claim 1, wherein the coupling element is preloaded between the first body and the second body in a starting position of the device, and wherein the first body is in contact with the second body.

10. The device as claimed in claim 6, wherein the one or more interruptions that are fluidically connected to the passage opening are configured to allow the flow of the filling medium through the second body, in an open valve position
